(19) United States
(12) Patent Application Publication
(10) Pub. No.: US 2002/0069883 A1
(43) Pub. Date: Jun. 13, 2002

(54) Device for active regulation of pressure on outer ear

(76) Inventor: Aviv Hirchenbain, Tel Aviv (IL)

Correspondence Address: DR. MARK FRIEDMAN LTD., c/o BILL POLKINGHORN- DISCOVERY DISPATCH, 9003 FLORIN WAY, UPPER MARLBORO, MD, 20772, US

(21) Appl. No.: 09/731,972

(22) Filed: Dec. 8, 2000

(51) Int. Cl. A61F 11/00

(52) U.S. Cl. 128/867; 128/864

(57) ABSTRACT

A device and method to protect a user's ear from changes in external atmospheric pressure and to treat middle ear disease by actively controlling (increasing, decreasing, oscillating or preserving unchanged) air pressure in the user's outer ear cavity. Preferably pressure is controlled using an earplug that fits tightly into the outer ear cavity with minimal air leakage. An axial channel through the earplug connects a pressure source which controls pressure to the outer ear cavity. Optionally the device also includes a conveyor and oscillator to supply controlled pressure oscillations to the outer ear cavity. A method to treat middle ear disease in a patient by transmitting vibrations to the patient's middle ear. Vibrations are transmitted by bone conduction or by pressure oscillations in the outer ear cavity. Vibrations reduce swelling and pain in the middle ear. The scope of the present invention includes use of the device for preventing pressure differential across the eardrum between the middle ear and the outer ear cavity and for the treatment of middle ear disease.

### FIELD AND BACKGROUND OF THE INVENTION

The present invention relates to a device and method to protect a user's ear from changes in external atmospheric pressure and to treat middle ear disease and more particularly a device and method to actively control (increase, decrease, oscillate or preserve unchanged) air pressure in the user's outer ear cavity and transmit vibrations to the user's middle ear.

The ear (FIG. 1) contains two cavities, an outer ear cavity 10 and a middle ear 12. An eardrum 14 separates the two cavities. If there is a difference in pressure between these two cavities there will be a stress on eardrum 14. This stress can cause pain, tissue damage or hearing loss.

Outer ear cavity 10 is in direct contact with an external atmosphere (not shown) while the only contact between middle ear 12 and the external atmosphere is through an Eustachian tube 16. Eustachian tube 16 is a narrow tube between middle ear 12 and a nasopharynx (not shown). Under normal conditions Eustachian tube 16 opens in response to yawning or swallowing. This preserves pressure equilibrium between middle ear 12 and the nasopharynx. The nasopharynx is in pressure equilibrium with the external atmosphere. Therefore, under normal conditions, middle ear 12 and outer ear cavity 10 are both in pressure equilibrium with the external atmosphere. Therefore the two cavities are in mutual pressure equilibrium and there is no stress on eardrum 14.

The pressure in outer ear cavity 10 immediately equilibrates to pressure changes in the external atmosphere. On the other hand, pressure changes in middle ear 12 require slow venting of air through narrow Eustachian tube 16. Therefore, sudden pressure changes in the external atmosphere may cause temporary pressure differential between outer ear cavity 10 and middle ear 12. This pressure differential stresses eardrum 14 and may result in pain (acute Barotrauma).

Barotrauma is often experienced by airplane passengers during landing and take off. For example, when an airliner ascends, the external pressure (cabin pressure) and the pressure in outer ear cavity 10 are immediately reduced. On the other hand, the reduction of pressure in middle ear 12 occurs much more slowly. This results in temporary over-pressure in middle ear 12. Until the excess pressure in middle ear 12 is fully vented, eardrum 14 will be stressed outward causing pain.

Eustachian tube 16 may become blocked in children or in adults with respiratory infections. This makes the equilibrating process between middle ear 10 and the external atmosphere exceedingly slow. On a long air journey, an air passenger with blocked Eustachian tube 16 may experience extreme and continuing pain due to pressure differential between outer ear cavity 10 and middle ear 12. Furthermore, this pressure differential can lead to chronic pain, hearing loss and tissue damage.

Mobley et al. (U.S. Pat. No. 5,467,784) developed a device that is intended to help airline passengers limit pressure differential between middle ear 12 and outer ear cavity 10. The Mobley et al. device is a passive earplug that shields outer ear cavity 10 from sudden pressure changes in the external atmosphere. The Mobley et al. device must be inserted into outer ear cavity 10 before any pressure change in the external atmosphere and must remain in outer ear cavity 10 continuously until pressure in middle ear 12 equilibrates with the pressure in the external atmosphere. If the Mobley et al. device is not inserted before pressure changes in the external atmosphere (take off) or if the Mobley et al. device falls out or is removed early then the Mobley et al. device gives no further benefit. Therefore the Mobley et al. device will fail for travelers who, at the beginning of their trip, were not aware of blockages that may exist in their Eustachian tube 16. The Mobley et al. device will also fail for travelers who prematurely remove the device. Furthermore, the Mobley et al. device can not be adjusted and retains pressure for a preset period that may not be appropriate for all users. Thus, for a passenger with blocked Eustachian tube 16, the preset pressure retention period of the Mobley et al. device may be too short. When the pressure retention period of the Mobley et al. device is too short, then the Mobley et al. device will not prevent Barotrauma. On the other hand the preset pressure retention period of the Mobley et al. device may be too long for passengers with clear Eustachian tube 16. When the pressure retention time of the Mobley et al. device is too long, then pressure in middle ear 12 will change faster in response to changes in external atmospheric pressure than pressure in outer ear cavity 10 causing (inverse) Barotrauma.

Another disadvantage of the Mobley et al. device is that it prevents pressure oscillations from reaching outer ear cavity 10. This reduces the hearing of the user of the Mobley et al. device. Reduced hearing causes inconvenience to the user who wishes to listen to music or join a conversation. Reduced hearing can also be a safety hazard because the user fails to receive important information such as announcements over an airplane's intercom. Furthermore, controlled low frequency pressure oscillations passed by eardrum 14 to middle ear 12 can be used therapeutically to reduce congestion of Eustachian tube 16 and swelling of tissue in middle ear 12.

Proetz (Proetz, A. W. “Allergy in middle and internal ear.” Ann Otol. 40: 67, 1931) developed a treatment for chronic blockage of Eustachian tube 16 by actively producing pressure oscillations (short-term overpressure) in the nasopharynx. Pressure in the nasopharynx affects middle ear 12 through Eustachian tube 16. Recently, Arick et al. (U.S. Pat. No. 5,419,762), Stangerup (U.S. Pat. No. 5,431,636) and Donaldson et al. (U.S. Pat. No. 5,950,631) developed devices to treat chronic ear diseases by controlling pressure in the nasopharynx. Controlling pressure in the nasopharynx can not alleviate Barotrauma in victims of blocked Eustachian tube 16 because the pressure from the nasopharynx can not traverse blocked Eustachian tube 16 to reach middle ear 12 fast enough to prevent pain or damage.

Liquid in middle ear 12 is common cause of reduced hearing in children. Liquid is drawn into middle ear 12 from surrounding tissue when there is negative pressure in middle ear 12. Negative pressure in middle ear 12 occurs when a child with partially blocked Eustachian tube 16 clears his nose by sucking. The Proetz methodology is not well suited for treating negative pressure in middle ear 12 of small children because applying pressure oscillations to the nasopharynx requires highly coordinated motor activity on the part of the user (stretching the neck and swallowing or blowing while holding the breath). It is desirable that there be a device to apply therapeutic pressure oscillations to middle ear 12 without requiring highly coordinated motor activity of the user.

Thus, there is a widely recognized need to prevent acute Barotrauma and associated long-term ear damage in people experiencing rapid external pressure changes. Barotrauma is particularly serious for people with blocked Eustachian tube 16. Barotrauma in a person with blocked Eustachian tube 16 will last longer than Barotrauma in a person with clear Eustachian tube 16. Furthermore, in a person with blocked Eustachian tube 16, Barotrauma will be have more serious complications and long term side effects than Barotrauma in a person whose Eustachian tube 16 is clear. Therefore, it is highly desirable to have a method to reverse pressure differentials between middle ear 12 and outer ear cavity 10 after the onset of Barotrauma (when a victim becomes aware of the Barotrauma due to perceived pain). It is further desirable that the method to reverse pressure differences between outer ear cavity 10 and middle ear 12 be effective also in the presence of blockage of Eustachian tube 16. It is further desirable that the method allows adjustable rate equilibration of pressure between outer ear cavity 10 and the external atmosphere and it is further desirable that there be further provided a procedure for conveying pressure oscillations and vibrations to the user's ear.

### SUMMARY OF THE INVENTION

According to the present invention there is provided a device for active control of air pressure in an outer ear cavity of a user's ear including: (a) a pressure source; and (b) an applicator to apply air pressure from the pressure source to the outer ear cavity.

According to the present invention there is provided a device for active control of air pressure in both outer ear cavities of two of a user's ears including: (a) a pressure source; and (b) an applicator to apply air pressure from the pressure source to the outer ear cavities.

According to the present invention there is provided an active method to prevent barotrauma in at least one of two ears of a user including the steps of: (a) providing a source of controlled pressure; and (b) applying the controlled pressure to an outer ear cavity of the at least one ear.

According to the present invention there is provided a method to treat ear disease in an ear of a patient including the steps of: (a) providing a source of controlled pressure; and (b) applying the controlled pressure to the patient's outer ear cavity.

According to the present invention there is provided a method to treat ear disease in an ear of a patient comprising the steps of: (a) providing a source of vibrations; and (b) transmitting the vibrations to tissue surrounding a middle ear cavity of the ear.

As understood herein, the terms pressure and air pressure include both positive gauge pressure and negative gauge pressure (suction).

According to further features in preferred embodiments of the invention described below, the device may also include a pressure conveyor. The conveyor serves to transmit pressure oscillations to outer ear cavity 10 of the user via the applicator. The pressure oscillations may be of an audible frequency or of an inaudible frequency. Audible oscillations include sounds from the external atmosphere, music or the contents of a sound channel (for example the audio program of an aircraft). Low frequency (inaudible) pressure oscillations have therapeutic value for users suffering from swelling or blockage in middle ear cavity 12 or Eustachian tube 16.

Unlike the Mobley et al. device, the present invention is active. Therefore, the present invention can be used to equilibrate pressure differentials between the outer ear cavity and middle ear after the onset of Barotrauma. The present invention can be adjusted during use allowing users with blocked Eustachian tube to choose a longer pressure equilibration time in the outer ear cavity and allowing users with clear Eustachian tube to choose a shorter equilibration time.

Unlike devices based on the Proetz methodology, the present invention controls pressure in the outer ear cavity and therefore prevents acute Barotrauma even in the presence of blocked Eustachian tube. Therefore the present invention functions in the situations described above where prior art devices fail.

Preferably, the user's outer ear cavity is insulated from pressure in the external atmosphere by means of an earplug. A channel is bored axially through the earplug and connected to a flexible tube. The flexible tube connects the outer ear cavity through the channel in the earplug to a pressure source (a source of increased or decreased pressure). The flexible tube also acts as a pressure buffer and preserves the pressure in the outer ear cavity when the pressure source is not active even in the presence of small leaks around the earplug.

### BRIEF DESCRIPTION OF THE DRAWINGS

The invention is herein described, by way of example only, with reference to the accompanying drawings, wherein:

FIG. 1 is a schematic view of an ear;

FIG. 2a is an axial cross section (taken through lines B-B of FIG. 2b) of a ribbed earplug of a preferred embodiment of the present invention;

FIG. 2b is a transverse cross section (taken through lines A-A of FIG. 2a) of a ribbed earplug of a preferred embodiment of the present invention;

FIG. 3 shows a preferred embodiment of the present invention with an independent ribbed earplug for each ear. Pressure is controlled using a removable pipette squeeze ball and flexible tubing;

FIG. 4 shows an alternative preferred embodiment of the present invention including two alternative embodiments of a conveyor which transmits pressure oscillations to outer ear cavity 10, and tapered earplugs held to the ear by a flexible assembly similar to a medical stethoscope. Pressure is supplied by a standard disposable medical syringe. A valve directs controlled pressure to each ear independently;

FIG. 5a is an axial cross section (taken through lines E-E of FIG. 5b) of an alternative embodiment of a conveyor which transmits pressure oscillations;

FIG. 5b is a transverse cross section (taken through lines D-D of FIG. 5a) of an alternative embodiment of a conveyor which transmits pressure oscillations;

FIG. 6 is a transverse cross section taken through the center of an alternate preferred embodiment of a pressure oscillation source assembly;

FIG. 7a is a transverse cross section (taken through lines F-F of FIG. 7b) of an alternate preferred embodiment of a pressure oscillation source assembly;

FIG. 7b is an axial cross section (taken through lines G-G of FIG. 7a) of an alternate preferred embodiment of a pressure oscillation source assembly;

FIG. 8a is a further alternate embodiment of a pressure source assembly;

FIG. 8b is a transverse cross section taken through the center of a 4-port 2-way valve used in the preferred embodiment of FIG. 8a configured for descent;

FIG. 8c is a transverse cross section taken through the center of a 4-port 2-way valve used in the preferred embodiment of FIG. 8a configured for ascent;

FIG. 9 is an illustration of the application of vibrations to the mastoid bone.

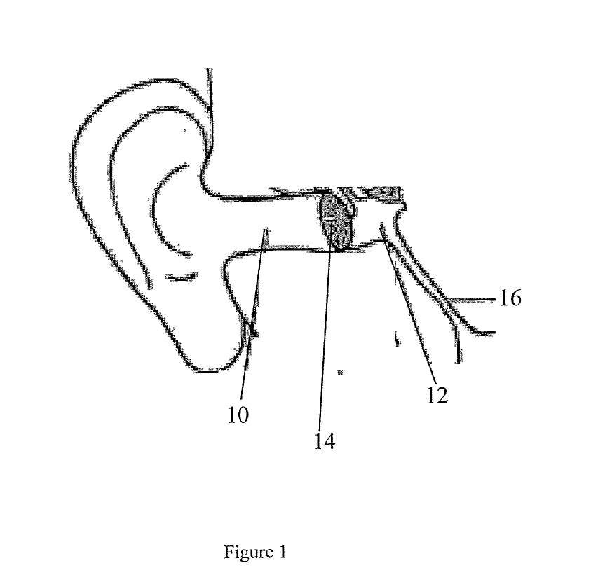

### DESCRIPTION OF THE PREFERRED EMBODIMENTS

The present invention is of a pressure controlling device. Specifically the pressure controlling device of the present invention serves for protecting a user's ear from changes in external atmospheric pressure and for treating middle ear disease by actively controlling (increasing, decreasing, oscillating or preserving unchanged) air pressure in the user's outer ear cavity.

The principles and operation of a pressure controlling device according to the present invention may be better understood with reference to the drawings and the accompanying description.

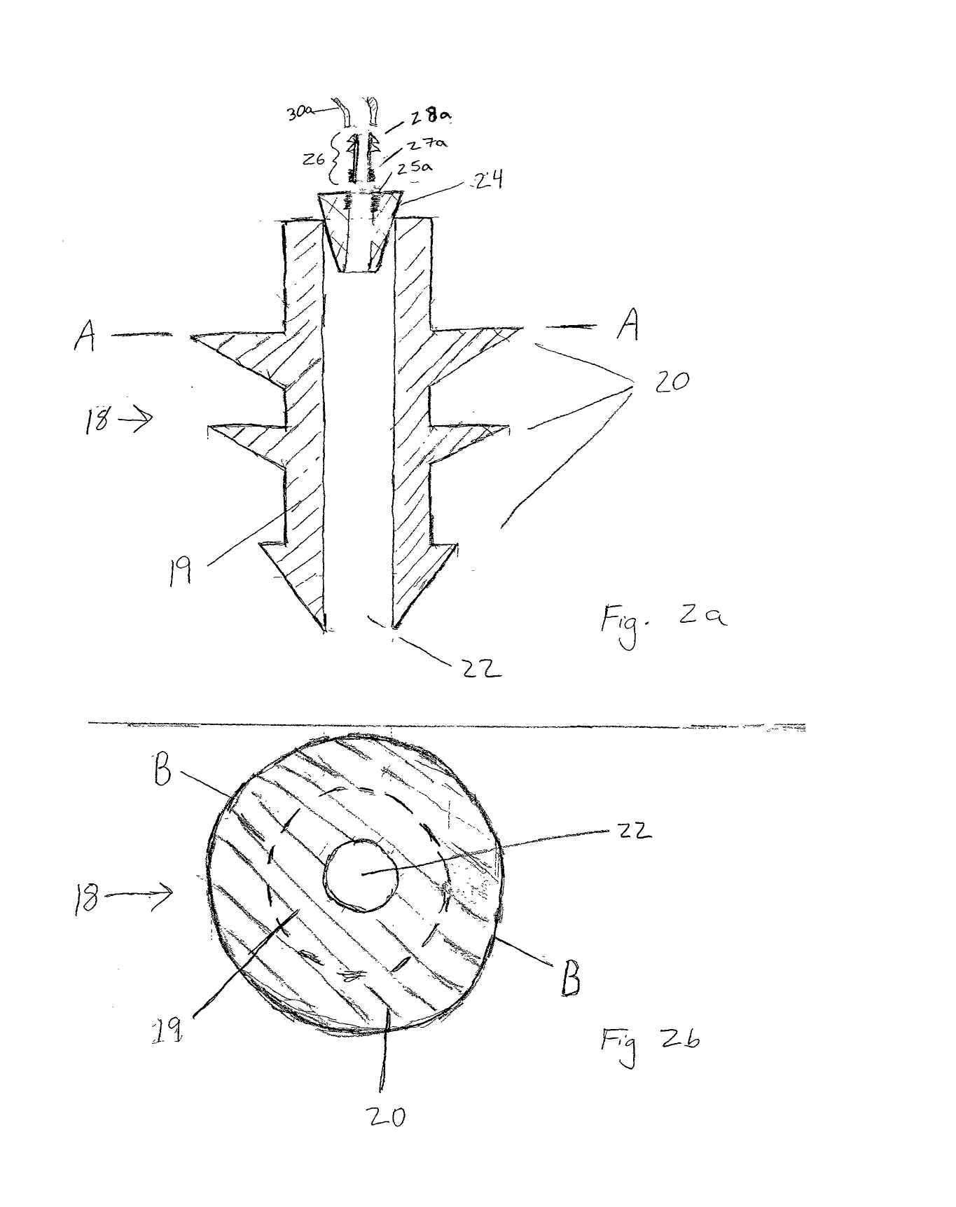

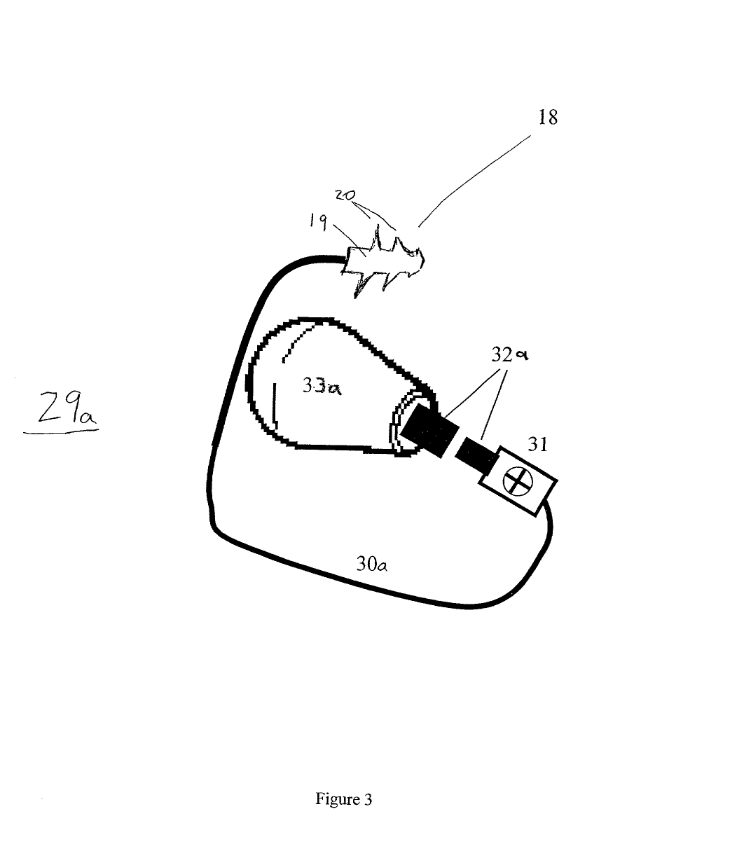

A preferred embodiment of the present invention is shown in FIGS. 2a, 2b and 3. FIGS. 2a and 2b respectively show detailed axial and transverse cross sections of an earplug 18. Earplug 18 has a solid cylindrical body 19 and ribs 20. Earplug 18 is a common sound attenuating earplug with the following modifications: an axial channel 22 is bored through earplug body 19. A conical fitting 24 with an internal thread 25a is inserted into channel 22. One end of a tubing adapter 26 screws into conical fitting 24 by means of an external thread 27a. The other end of tubing adapter 26 connects by means of a barbed tube fitting 28a to a flexible tube 30a.

When earplug 18 is inserted into outer ear cavity 10, outer ear cavity 10 is insulated from pressure in the external atmosphere by ribs 20 while flexible tube 30a and channel 22 provide a path for control of pressure within outer ear cavity 10.

FIG. 3 shows an entire preferred embodiment 29a of the present invention. Flexible plastic tube 30a connects ribbed earplug 18 to an on-off valve 31 that can be opened and closed. Ribbed earplug 18 has a cylindrical body 19 and ribs 20. On-off valve 31 is connected by means of a standard plastic tubing quick connect friction fitting 32a to a pressure source 33a which, in embodiment 29a is a standard pipette squeeze ball.

When the user (e.g., a passenger in an ascending airplane) feels discomfort due to overpressure in middle ear 12, the user inserts earplug 18 into outer ear cavity 10 and uses friction quick connector 32a to attach pressure source 33a to on-off valve 31. The user then opens on-off valve 31 and squeezes pressure source 33a. This increases the pressure in outer ear cavity 10 relieving the pressure differential between middle ear 12 and outer ear cavity 10. The user may then close on-off valve 31 and remove pressure source 33a (leaving earplug 18 in outer ear cavity 10) until the user feels a need for further equilibration. With on-off valve 31 closed, a large volume of air in flexible tube 30a acts as a buffer retaining the pressure inside outer ear cavity 10 even in the presence of small leaks around earplug 18.

When the user (e.g. a passenger in a descending airplane) feels discomfort due to under-pressure in middle ear 12, the user inserts earplug 18 into outer ear cavity 10. Then the user squeezes pressure source 33a collapsing pressure source 33a and emptying pressure source 33a of air. The user then attaches collapsed pressure source 33a to on-off valve 31 using friction quick connector 32a. Finally, the user opens on-off valve 31 and releases pressure source 33a allowing pressure source 33a to resume its natural (inflated) shape producing a vacuum. This reduces the pressure in outer ear cavity 10 relieving the pressure differential between middle ear 12 and the outer ear cavity 10. The user may then close on-off valve 31 and remove pressure source 33a (leaving earplug 18 in outer ear cavity 10) until the user feels a need for further equilibration. With on-off valve 31 closed, a large volume of air in flexible tube 30a acts as a buffer retaining the pressure inside outer ear cavity 10 even in the presence of small leaks around earplug 18.

An alternative preferred embodiment of the present invention, which is referred to herein as system 29b, is shown in FIG. 4. Tapered earplugs 35 are sealed over outer ear cavities 10 of both of the user's ears using a frame 36 (similar to a doctor's stethoscope or to audio earphones). Each earplug 35 contains an axial channel (not shown) which is connected to four-way valve 38 by means of flexible tubing 30b. Pressure source 33b is connected to four-way valve 38. Four-way valve 38 allows the user to close off tube 30b or to connect tube 30b to pressure source 33b or to the external atmosphere. Four-way valve 38 allows separate control of the pressure in outer cavity 10 of each ear. Pressure source 33b in this alternate embodiment is a standard disposable medical syringe which connects to four-way valve 38 by means of a standard threaded hypodermic quick connect 32b. Pressure in outer ear cavity 10 can by increased by using pressure source 33b to inject air into flexible tubing 30b. Pressure in outer ear cavity 10 can by decreased by using pressure source 33b to extract air from flexible tubing 30b.

Flexible tubing 30b includes a safety pressure release 41. Safety release 41 in this embodiment is a thinning of the walls of flexible tubing 30b. In the event of dangerously high pressures within tubing 30b, thinned safety release 41 bulges to release pressure. In the event of dangerously low pressures within tubing 30b, thinned safety release 41 collapses to prevent sudden release of pressure from outer ear cavity 10. Alternatively safety release 41 may be a pressure release valve.

Flexible tube 30b further includes a constriction 42 which prevents large volumes of air from quickly entering or leaving the channel of earplug 35. Thus the air in the hollow of flexible tubing 30b between earplug 35 and constriction 42 acts as a buffer protecting outer ear cavity 10 from sudden pressure changes. Alternatively, the channel of earplug 35 could itself be narrow and serve as a constriction; the small volume of air inside of outer ear cavity 10 serving as a pressure buffer.

Flexible tubing 30b also includes a conveyor 44a which in this embodiment is a standard tubing Y-connect. In this preferred embodiment, conveyor 44a is to be connected to a pressure oscillation source assembly 56a. Pressure oscillation source assembly 56a includes a miniature loudspeaker 48a (similar to the miniature earphone speakers included with portable cassette players) which is connected by a wire 45a which passes through stopper 50 to a standard audio earphone plug 46a. Loudspeaker 48a is inserted through conveyer 44a into the hollow of tubing 30b and stopper 50 is inserted into the opening of conveyor 44a preventing pressure leakage to the external atmosphere. Plug 46a can be inserted into an aircraft sound system plug or a portable tape player to allow the user to listen to music or plug 45a can be attached to a microphone (not shown) to allow the user to better to hear sounds in the external atmosphere.

System 29b further includes an alternative preferred embodiment of a conveyor 44b. Conveyor 44b is a diaphragm assembly communicating pressure oscillations to the cavity of flexible tube 30b through a barbed tubing connector 28b. Diaphragm assembly conveyor 44b includes a rigid cylindrical box 60a which has an open end covered by a flexible diaphragm 62. As shown, diaphragm assembly conveyor 44b transmits pressure oscillations from the external atmosphere via the internal cavity of tube 30b to outer ear cavity 10 improving the ability of the user to hear sounds in the external atmosphere. Diaphragm assembly conveyor 44b also includes an external thread 27b to connect diaphragm assembly conveyor 44b to a pressure oscillation source.

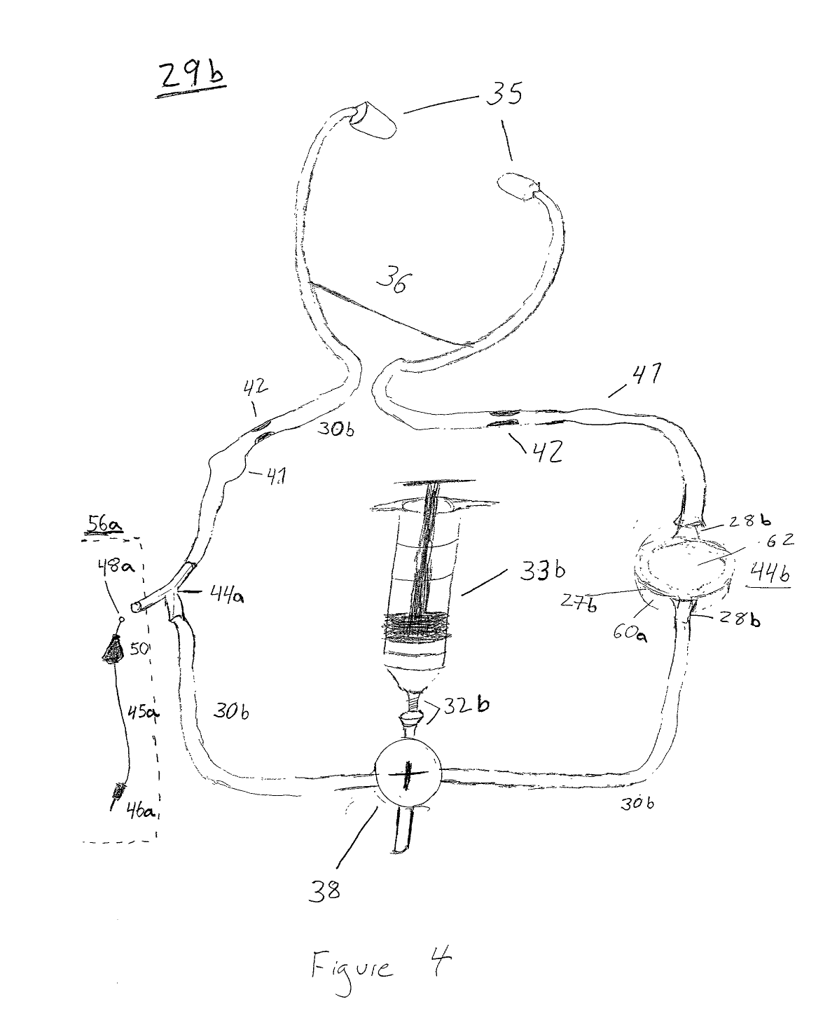

Diaphragm assembly conveyor 44b and barbed tube connectors 28b are shown in more detail in axial cross section in FIG. 5a and in transverse cross section in FIG. 5b. Diaphragm assembly conveyor 44b includes rigid cylindrical box 60a, which has an open end covered by flexible diaphragm 62. In this preferred embodiment diaphragm assembly conveyor 44b has an external thread 27b to connect diaphragm assembly conveyor 44b to a pressure oscillation source. Also shown in FIG. 5b is a transverse cross section through the center of a connector 66, which connects diaphragm assembly conveyor 44b to a pressure oscillation source. Connector 66 is a rigid cylindrical box 60b open at one end with an internal thread 25b which screws connector 66 (like a jar cover) to diaphragm assembly conveyor 44b. A barbed tube fitting 28c serves to join connector 66 to a pressure oscillation source.

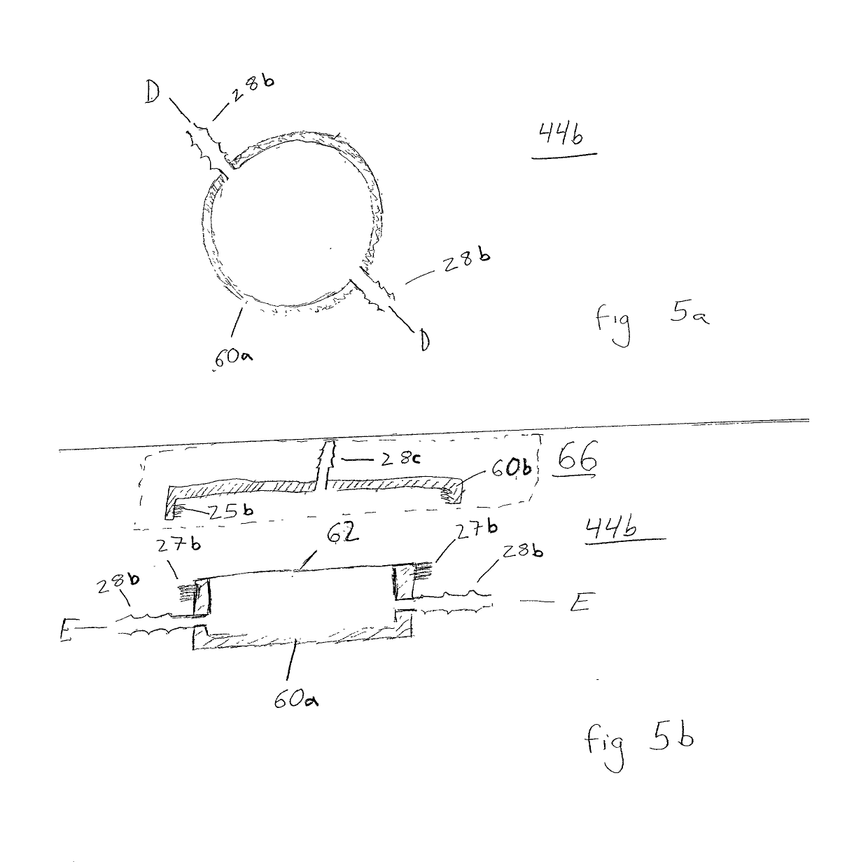

A transverse cross section through the center of an alternative preferred embodiment of a pressure oscillation source assembly, which is referred herein as assembly 56b is shown in FIG. 6. Assembly 56b contains a rigid cylindrical box 60c in which is mounted a standard audio speaker 48b connected to an oscillating electric current by means of wire 45b and audio earphone plug 46b. Pressure oscillations of loudspeaker 48b are transmitted via barbed tubing connector 28d and flexible tubing 30c to a conveyor (for example 44a or 44b). An oscillating electric current may be applied to earphone plug 46b from any audio device, for example a tape deck (not shown) or the sound system of an aircraft (not shown).

Low frequency pressure oscillations (low frequency sounds or sub-audio low frequency vibrations) have a therapeutic effect reducing swelling and pain in middle ear 12 and Eustachian tube 16. Thus, therapeutic treatment of middle ear disease (swelling in middle ear 12 or blocking of Eustachian tube 16) is achieved by placing pressure regulation device 29b over the ears of a patient and supplying low frequency pressure oscillations. Such pressure oscillations are supplied by oscillation source assembly 56b which is connected by means of audio plug 46b to a tape player (not shown) playing a custom audio tape of low frequency signals (not shown).

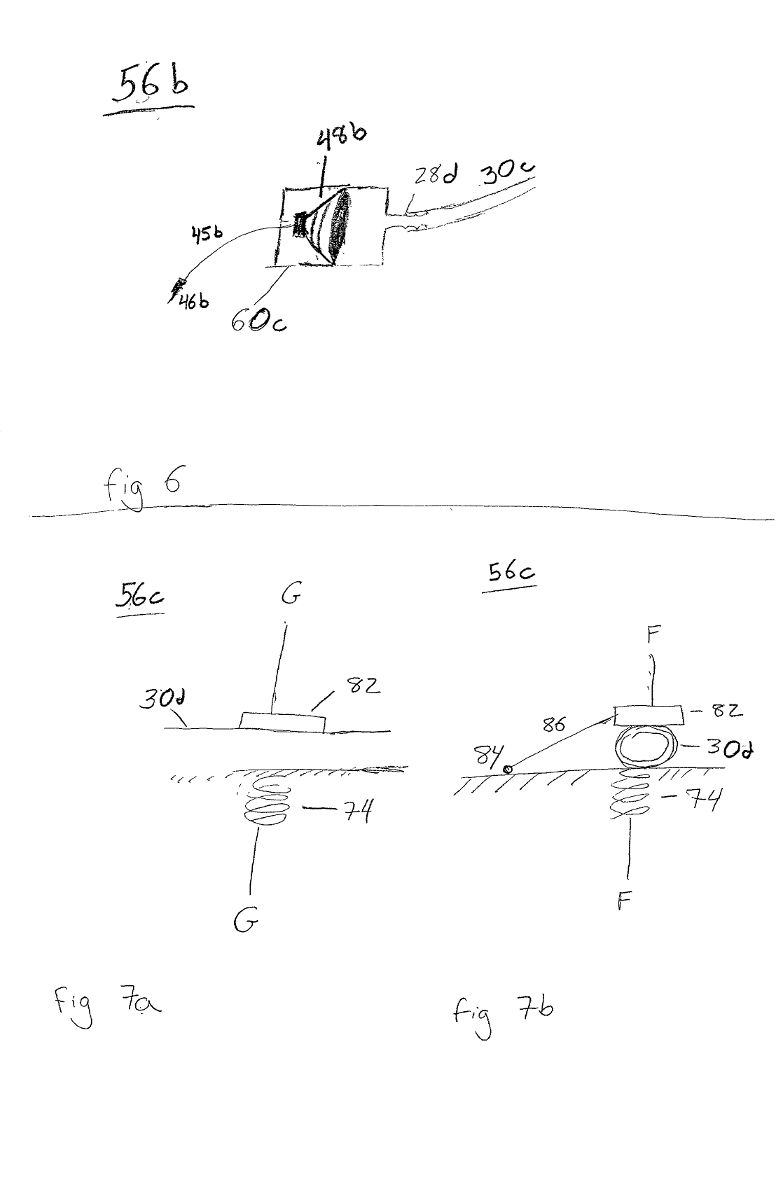

Another alternate embodiment of a pressure oscillation source referred to herein as assembly 56c is shown in FIGS. 7a and 7b. Assembly 56c includes an electromagnet 74, which pulls down a press 82 which is mounted on an actuator arm 86 connected to a hinge 84. When electric current is applied to magnet 74, press 82 squeezes flexible tubing 30d reducing the internal volume of tubing 30d. When the ends of tubing 30d are sealed, decreasing the volume of tube 30d increases internal pressure. For example, assembly 56c may be installed along tubing 30b that is included in the applicator apparatus of FIG. 4. Applying an oscillating current to magnet 74 creates pressure oscillations inside tube 30d which are translated to outer ear cavity 10. Assembly 56c can produce very low frequency pressure oscillation similar to pressure oscillations produced by muscular activity in the Proetz methodology. Thus, assembly 56c in combination with applicator assembly 29b applies therapeutic pressure oscillations to outer ear cavity 10 alleviating swelling of middle ear 12 and blockage of Eustachian tube 16.

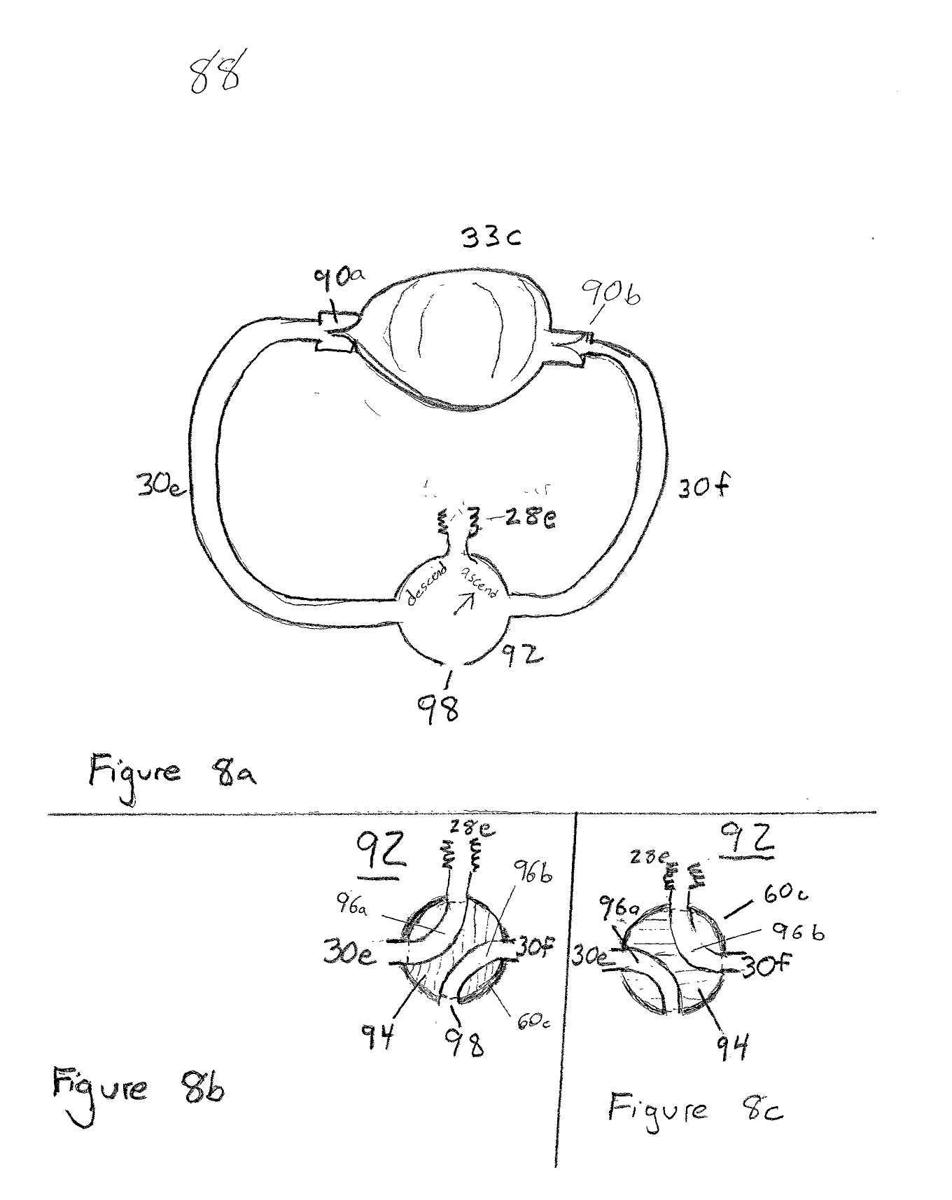

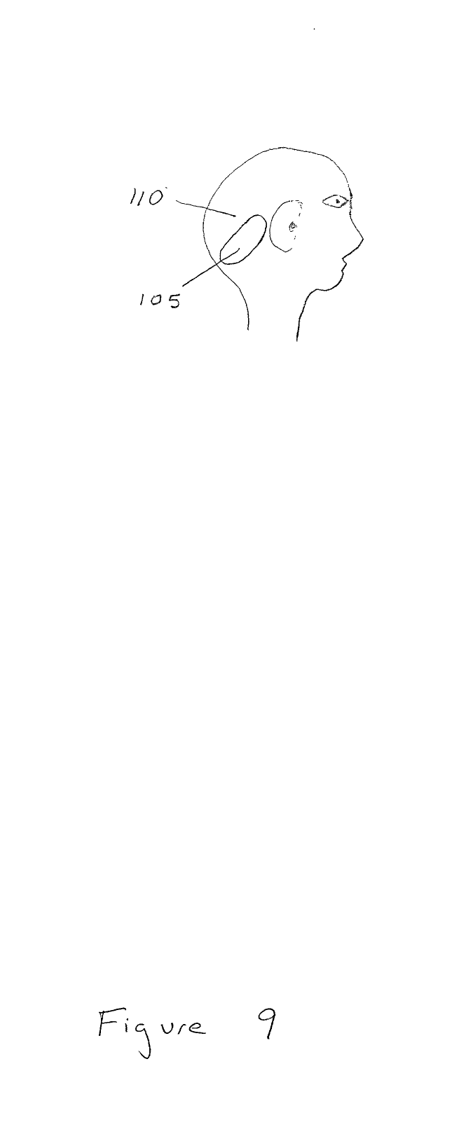

FIG. 8a shows an alternative embodiment of a pressure source assembly herein referred to as pressure source assembly 88. Squeezing pressure source 33c, which is a squeeze ball in this preferred embodiment, pushes air through a one-way valve 90a into a flexible tube 30e. Releasing pressure source 33c from its collapsed state allows source 33c to inflate drawing air through a one-way valve 90b from a flexible tube 30f.

Flexible tubes 30e and 30f are further connected to two ports of a four-port two-way valve 92. Four-port two-way valve 92 is further connected to an applicator (not shown) via a barbed tube fitting 28e and to an external atmosphere (not shown) via an exit port 98. A transverse cross section of four-port two-way valve 92 is shown in FIG. 8b and FIG. 8c. Four-port two-way valve 92 includes a cylindrical body 60c and a rotating inner chamber 94. Rotating inner chamber 94 contains two channels 96a and 96b.

Four-port two-way valve 92 has two configurations (ways): The first configuration herein referred to as descent is shown in FIG. 8b. In the descent configuration, channel 96a connects flexible tube 30e to the applicator via barbed tube fitting 28e. Simultaneously, in the descent configuration, channel 96b connects flexible tube 30f to the external atmosphere via exit port 98. Thus when four-port two-way valve 92 is in the descent configuration, alternately squeezing and releasing pressure source 33c draws air through exit port 98 and pumps the air into the applicator, increasing pressure on the outer ear cavity. The second configuration herein referred to as ascent is shown in FIG. 8c. In the ascent configuration, channel 96a connects flexible tube 30e to the external atmosphere exit port 98. Simultaneously, in the ascent configuration, channel 96b connects flexible tube 30f to the applicator via barbed tube fitting 28e. Thus when four-port two-way valve 92 is in the ascent configuration, alternately squeezing and releasing pressure source 33c vents air through exit port 98 and draws the air out of the applicator decreasing pressure on outer ear cavity.

In FIG. 9 a commercially available muscle relaxation vibrator 105 is shown being applied to the mastoid bone 110 of a patient. Vibrations relieve middle ear disease due to Eustachian blockage because vibrations reduce swelling in middle ear tissue by inducing blood flow. Vibrations also break up blockages in Eustachian tube 16 and reduce pain through competitive nerve stimulation.

It will be appreciated that the above descriptions are intended only to serve as examples, and that many other embodiments are possible within the spirit and the scope of the present invention.

What is claimed is

1. A device for active control of air pressure in an outer cavity of a user's ear comprising:
a) a pressure source; and
b) an applicator to apply air pressure from said pressure source to the outer ear cavity.

2. The device of claim 1, wherein said applicator is operative to insulate the outer ear cavity from pressure changes in the external atmosphere.

3. The device of claim 2, wherein said applicator includes an earplug having an axial channel such that when said earplug is inserted in the outer ear cavity, air enters and leaves the outer ear cavity substantially only via said channel, said channel then conducting pressure between said pressure source and the outer ear cavity.

4. The device of claim 2, wherein said applicator further includes a safety pressure release that limits a pressure difference between the outer ear cavity and an external atmosphere.

5. The device of claim 2, wherein said applicator further includes a constriction to impede sudden changes of pressure in the outer ear cavity.

6. The device of claim 2, wherein said applicator further includes a volume of dead air space acting as a pressure buffer preserving pressure in said applicator.

7. The device of claim 1, wherein pressure output of said pressure source is adjustable by the user.

8. The device of claim 1, wherein said pressure source is manually operated by the user.

9. The device of claim 1, wherein said pressure source includes a squeeze ball.

10. The device of claim 1, wherein said pressure source includes a syringe.

11. The device of claim 1, further comprising:
c) at least one quick connector for reversibly connecting said pressure source to said applicator.

12. The device of claim 1, wherein said applicator includes at least one valve for reversibly admitting said air pressure from said pressure source to the outer ear cavity.

13. The device of claim 1, wherein said applicator further includes a frame securing said pressure applicator to the outer ear cavity of the user.

14. The device of claim 1, further comprising:
c) a conveyor for transmitting pressure oscillations via said applicator to the outer ear cavity.

15. The device of claim 14, further comprising:
d) a pressure oscillation source supplying said pressure oscillations to said conveyor.

16. A device for active control of air pressure in outer cavities of both of a user's ears comprising:
a) a pressure source; and
b) an applicator to apply pressure from said pressure source to the outer ear cavities of the user.

17. The device of claim 16, wherein said applicator includes at least one valve for reversibly admitting said air pressure from said pressure source to the outer cavity of each of the user's ears independently.

18. The device of claim 16, wherein said applicator further includes a frame for securing said pressure applicator to the outer ear cavities of the ears.

19. An active method to prevent barotrauma in at least one of two ears of a user comprising the steps of:
a) providing a source of controlled pressure;
b) applying said controlled pressure to an outer ear cavity of the at least one ear.

20. The method of claim 19, further comprising the step of:
c) insulating the outer ear cavity of the at least one ear from pressure changes in the external atmosphere.

21. The method of claim 19, wherein said applying of pressure is effected by the user.

22. The method of claim 19, wherein said controlled pressure includes pressure oscillations.

23. A method to treat ear disease in an ear of a patient comprising the steps of:
a) providing a source of controlled pressure; and
b) applying said controlled pressure to the patient's outer ear cavity.

24. The method of claim 22, further comprising the step of:
c) insulating the user's outer ear cavity from pressure in the external atmosphere.

25. The method of claim 22, wherein said controlled pressure includes pressure oscillations.

26. A method to treat ear disease in an ear of a patient comprising the steps of:
a) providing a source of vibrations; and
b) transmitting said vibrations to ear tissue of the ear.

27. The method of claim 26, wherein said step of transmitting vibration to tissue is accomplished by applying tremors to a bone, said tremors being transmitted to said tissue via bone conduction.

28. The method of claim 27, wherein said bone is a mastoid bone.
